(19) United States
(12) Patent Application Publication
(10) Pub. No.: US 2021/0052473 A1
(43) Pub. Date: Feb. 25, 2021

(54) COMPOSITION FOR REMOVING ALDEHYDE-BASED ODORS AND REACTIVE OXYGEN SPECIES

(71) Applicant: Osteon Co. Ltd., Daejeon (KR)

(72) Inventors: Sang Baek Kim, Daejeon (KR); Sung Soo Kim, Daejeon (KR)

(73) Assignee: Osteon Co. Ltd., Daejeon (KR)

(21) Appl. No.: 16/996,593

(22) Filed: Aug. 18, 2020

(30) Foreign Application Priority Data
Aug. 21, 2019 (KR) 10-2019-0102204

(51) Int. Cl. A61K 8/23; A61Q 15/00; A61K 8/44; A61K 8/46; A61K 8/60; A61K 8/49; A23L 5/20; A23L 29/00; A23L 33/13; A23L 33/175

(52) U.S. Cl. CPC A61K 8/23; A61Q 15/00; A61K 8/44; A61K 8/466; A61K 8/606; A61K 8/4953; A23V 2002/00; A23L 5/276; A23L 29/015; A23L 33/13; A23L 33/175; A61K 2800/592; A61K 8/4946

(57) ABSTRACT

Disclosed are a composition for removing aldehyde-based odors and reactive oxygen species, and more particularly to a deodorizing composition for removing hydrophobic aldehyde-based odors, and a food additive or cosmetic additive containing the same as an active ingredient, wherein the deodorizing composition has an effect of removing reactive oxygen species and an effect of removing a substance causing an aldehyde-based odor, particularly a hydrophobic aldehyde-based odor, thus being useful as a cosmetic additive for reducing body odors from humans or animals and for providing an anti-aging function as well as a food additive for removing odors from food.

### CROSS-REFERENCE TO RELATED APPLICATION

This application claims under 35 U.S.C. § 119(a) the benefit of priority to Korean Patent Application No. 10-2019-0102204 filed on Aug. 21, 2019, the entire contents of which are incorporated herein by reference.

### BACKGROUND

### (a) Technical Field

The present invention relates to a composition for removing aldehyde-based odors and reactive oxygen species, and more particularly to a deodorizing composition for removing hydrophobic aldehyde-based odors, and a food additive or cosmetic additive containing the same as an active ingredient.

### (b) Background Art

Frequently encountered odors in daily life include aldehyde compounds produced by oxidation of unsaturated fatty acids. Foods having these compounds as the cause of odors, include fish, omega-3, squalene and the like, body odors generated by humans and animals are also derived from aldehyde compounds generated by oxidation of fatty acids in the skin and these aldehyde compounds include butanal, pentanal, hexanal, heptanal, octanal, decanal, nonenal and the like. These aldehyde compounds are produced by oxidation of unsaturated fatty acids, as in the example of FIG. 1. Once unsaturated fatty acids are bonded to oxygen to produce peroxides (reactive oxygen species), then chemical bonds are broken to produce aldehyde substances.

Aldehyde compounds including acetaldehyde are known carcinogens. Cellular experiments have shown that nonenal, which is a major component of age-related body odor and is a kind of aldehyde compound, also damages and kills cells and affects DNA modification. Nonenal is found in the skin after 40 years of age, and the amount thereof increases with age. This tendency is broadly consistent with the tendency of rapidly increasing cancer incidence after 40 years of age, and nonenal may therefore be predicted to be involved in cancer occurrence. In addition, nonenal causes strong eye irritation, and the onset of presbyopia, which appears in the early 40s, may be associated with this substance. That is, despite being present in small amounts, aldehyde compounds produced in the skin are inevitably inhaled by humans every day for a long time, which may cause the cancer occurrence in humans. In addition, reactive oxygen radicals in the form of aldehyde precursors may also cause food deterioration, carcinogenesis, and aging. Thus, it will be beneficial to health to remove reactive oxygen radicals from food and skin.

Other substances, such as alcohols, aldehydes, ammonia, amines, acetic acid, lactic acid, hydrogen sulfide and mercaptans, which are produced by enzymatic decomposition of microorganisms, are also major components of odors.

Because such odors are unpleasant to people, technologies to remove odors using various methods have been devised. Korean Patent No. 10-1075413 discloses a technique for removing nonenal using plant extracts such as Curcuma longa, pine needles and Scutellariae baicalensis, and Korean Patent No. 10-1260371 discloses a deodorizing composition for removing an ammonia odor using a mulberry root, a cypress extract, or the like. In addition, Korean Patent No. 10-1554537 discloses a composition for removing nonenal using a Castanea crenata inner shell extract, and Korean Patent No. 10-1846032 discloses a method for removing nonenal using polyphenols derived from plants. Meanwhile, Korean Patent Laid-open Publication No. 10-2015-0065279 discloses a method for removing nonenal using seaweed and a cypress extract. The methods using the plant extracts use aromas and antioxidation of polyphenols. Chemically, polyphenols may have a slight effect on oxidation of fatty acids, but with such a method it is difficult to remove the generated odor-inducing substance, including aldehydes such as nonenal.

Hydrophilic aldehydes such as formaldehyde, ammonia, amines, acetic acid, lactic acid, hydrogen sulfide, mercaptans and the like are hydrophilic and participate in acid/base reactions, and thus can be easily removed from air or water. On the other hand, hydrophobic aldehydes such as nonenal have oil-like properties and are not easy to remove. Thus, there is a need to remove hydrophobic aldehydes. In addition, since reactive oxygen species produced by unsaturated fatty acids in the human skin may cause cancer and accelerate aging, the removal of reactive oxygen species is considerably essential and useful in the fields such as cosmetics and food.

Accordingly, in the present invention, a method for removing hydrophobic aldehyde compounds such as nonenal and reactive oxygen species was developed, and more particularly, a biocompatible composition for efficiently removing hydrophobic aldehyde compounds was developed using only cosmetic or food raw materials, permitted for application to fields closely related to the human body, such as those of cosmetics and food ingredients.

### PRIOR ART DOCUMENT

### Patent Documents

### (Patent Document 1) Korean Patent No. 10-1075413

### (Patent Document 2) Korean Patent No. 10-1260371

### (Patent Document 3) Korean Patent No. 10-1554537

### (Patent Document 4) Korean Patent No. 10-1846032

### (Patent Document 5) Korean Patent Laid-open Publication No. 10-2015-0065279

### SUMMARY OF THE DISCLOSURE

The present invention has been made in an effort to solve the above-described problems associated with the prior art, and it is one object of the present invention to provide a deodorizing composition containing a first component including one or more sulfites and a second component including an amine compound having one or more amino groups as active ingredients.

It is another object to provide a food additive containing the deodorizing composition as an active ingredient.

It is another object to provide a cosmetic additive containing the deodorizing composition as an active ingredient.

It is another object to provide a method for removing aldehyde-based odors or reactive oxygen species using the deodorizing composition.

It is another object to provide a method for removing aldehyde-based odors including applying the deodorizing composition to or around a subject in need of odor removal.

The objects of the present invention are not limited to those described above. The objects of the present invention will be clearly understood from the following description and can be implemented by means defined in the claims and combinations thereof.

In one aspect, the present invention provides a deodorizing composition including, as active ingredients, a first component comprising at least one sulfite compound and a second component comprising at least one biological amine compound having one or more amino groups.

In an embodiment of the present invention, the first component may include at least one selected from the group consisting of sodium bisulfite, potassium bisulfite, lithium bisulfite, sodium metabisulfite, potassium metabisulfite, sodium metabisulfite, lithium metabisulfite, sodium hydrosulfite, potassium hydrosulfite, and lithium hydrosulfite.

In an embodiment of the present invention, the second component may include at least one selected from the group consisting of taurine, adenosine, cytosine, thymine, adenine, guanine, lysine, arginine, and histidine.

In an embodiment of the present invention, the first component and the second component may react with each other, and thus may be present as R₁—NHSO₂H.

In an embodiment of the present invention, the first component and the second component may be mixed at a molar ratio of 1:1 to 1.5.

In an embodiment of the present invention, the deodorizing may be removal of an aldehyde-based odor.

In an embodiment of the present invention, the aldehyde-based odor may be a hydrophobic aldehyde-based odor.

In an embodiment of the present invention, the substance causing the hydrophobic aldehyde-based odor may include at least one selected from the group consisting of butanal, pentanal, nonenal, hexanal, heptanal, octanal, decanal, and nonanal.

In an embodiment of the present invention, the deodorizing composition may have the ability to remove reactive oxygen species.

In another aspect, the present invention provides a food additive containing the deodorizing composition as an active ingredient.

In another aspect, the present invention provides a cosmetic additive containing the deodorizing composition as an active ingredient.

In another aspect, the present invention provides a method for removing aldehyde-based odors or reactive oxygen species using the deodorizing composition.

In another aspect, the present invention provides a method for removing aldehyde-based odors including applying the deodorizing composition to or around a subject in need of odor removal.

Other aspects and preferred embodiments of the invention are discussed Below. The above and other features of the invention are discussed Below.

### BRIEF DESCRIPTION OF THE DRAWINGS

The above and other features of the present invention will now be described in detail with reference to certain exemplary embodiments thereof, illustrated in the accompanying drawings which are given hereinbelow by way of illustration only, and thus are not limitative of the present invention, and wherein:

FIG. 1 shows an example of the chemical mechanism for producing nonenal, which is an aldehyde-involved compound produced by oxidation of an unsaturated fatty acid on the skin;

FIG. 2 is a schematic diagram showing the aldehyde-removing mechanism of the chemical reaction between an aldehyde-involved compound, and the first (sulfite) and second (amino) components for deodorizing according to an embodiment of the present invention;

FIG. 3 is the mechanism of the chemical reaction in the present invention, compared with Schiff base and Sulfite addition reactions; and

FIG. 4 is a schematic diagram showing the chemical mechanism for removing reactive oxygen species through the chemical reaction between peroxide-involved compounds produced by unsaturated fatty acids and the first (sulfite) and second (amine) components contained as active ingredients in the deodorizing composition according to an embodiment of the present invention.

### DETAILED DESCRIPTION

Unless the context clearly indicates otherwise, all numbers, figures and/or expressions that represent ingredients, reaction conditions, polymer compositions and amounts of mixtures used in the specification are approximations that reflect various uncertainties of measurement occurring inherently in obtaining these figures, among other things. For this reason, it should be understood that, in all cases, the term “about” should modify all numbers, figures and/or expressions. In addition, when numerical ranges are disclosed in the description, these ranges are continuous and include all numbers from the minimum to the maximum including the maximum within the range unless otherwise defined. Furthermore, when the range refers to an integer, it includes all integers from the minimum to the maximum including the maximum within the range, unless otherwise defined.

It should be understood that, in the specification, when a range is referred to regarding a parameter, the parameter encompasses all figures including end points disclosed within the range. For example, the range of “5 to 10” includes figures of 5, 6, 7, 8, 9, and 10, as well as arbitrary sub-ranges, such as ranges of 6 to 10, 7 to 10, 6 to 9, and 7 to 9, and any figures, such as 5.5, 6.5, 7.5, 5.5 to 8.5 and 6.5 to 9, between appropriate integers that fall within the range. In addition, for example, the range of “10% to 30%” encompasses all integers that include numbers such as 10%, 11%, 12% and 13% as well as 30%, and any sub-ranges of 10% to 15%, 12% to 18%, or 20% to 30%, as well as any numbers, such as 10.5%, 15.5% and 25.5%, between appropriate integers that fall within the range.

Also, the terms and abbreviations used herein may be interpreted as having meanings commonly understood by those skilled in the art to which the present invention pertains unless otherwise defined.

Hereinafter, the present invention will be described in detail.

The present invention relates to a composition for removing aldehyde-based odors and reactive oxygen species, and more particularly to a deodorizing composition for removing hydrophobic aldehyde-based odors, and a food or cosmetic additive containing the same as an active ingredient.

In one aspect, the present invention provides a deodorizing composition containing a first component comprising at least one sulfite compound and a second component comprising at least one biological amine compound having one or more amino groups.

The term “deodorization” refers to the removal of odors, and means removing, reducing or blocking odors, which are irritant gaseous substances causing discomfort or repulsion in humans.

As used herein, the term “odor” is defined as an odor of hydrogen sulfide, mercaptan, amine, or other irritating gaseous substances that stimulate the human sense of smell and cause discomfort and disgust according to the Air Environment Preservation Act. There are different types of substances, and these substances vary slightly depending on combined actions between malodorous substances, individual age, gender, psychological factors and body condition.

In the present invention, the deodorizing composition includes two active ingredients, that is, the first component including at least one sulfite compound and the second component including at least one biological amine compound having an amino group.

The “sulfite” included in the first component is lithium, sodium or potassium salt of sulfite, and is a substance used in the form of a food or cosmetic additive as a bleaching agent, preservative or antioxidant.

The first component includes, but is not limited to, at least one selected from the group consisting of sodium bisulfite, potassium bisulfite, lithium bisulfite, sodium metabisulfite, potassium metabisulfite, sodium metabisulfite, lithium metabisulfite, sodium hydrosulfite, potassium hydrosulfite, and lithium hydrosulfite.

The “amine compound”, “compound having one or more amino groups” or “amine compound having one or more amino groups” included in the second component refers to an organic compound in which a hydrogen atom of ammonia is substituted with a hydrocarbon group such as an alkyl group, and is a derivative of ammonia in which the hydrogen atom of ammonia is substituted with a substituent such as an alkyl group or an aryl group. The expression “compound having one or more amino group” may be used interchangeably with “amine compound”, and the amine compound may be classified into a primary amine (RNH₂), a secondary amine (R₂NH) and a tertiary amine (R₃N) depending on the number of substituted hydrogen atoms, and examples of essential amines include amino acids, bioamines, trimethylamine, aniline and the like.

In the present invention, examples of compounds having one or more amino groups included in the second component are primary or secondary amine compounds having little or no odor or toxicity, derived from an organism, and amino acids containing an amino group (a molecule in which one nitrogen atom and two hydrogen atoms are bonded to each other) and a carboxyl group (a molecule in which one carbon atom, two oxygen atoms and one hydrogen atom are bonded to one another), or biological materials such as nucleotides that form DNA.

The second component, without being limited thereto, has the following chemical structures, including at least one selected from the group consisting of taurine, adenosine, cytosine, thymine, adenine, guanine, lysine, arginine, and histidine.

| Second component | Chemical structure |
|---|---|
| taurine | 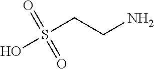 |
| adenosine | 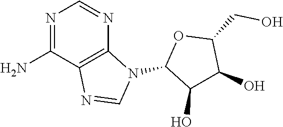 [SMILES: Nc1ncnc2c1ncn2[C@@H]1O[C@H](CO)[C@@H](O)[C@H]1O] |
| cytosine | 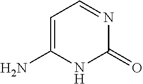 |
| thymine | 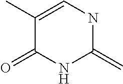 |
| adenine | 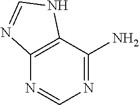 |
| guanine | 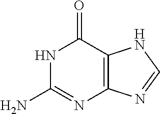 [SMILES: Nc1nc2nc[nH]c2c(=O)[nH]1] |
| lysine | 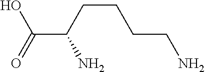 |
| arginine | 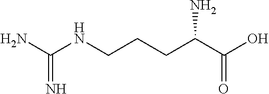 [SMILES: N=C(N)NCCC[C@H](N)C(=O)O] |
| histidine | 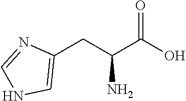 [SMILES: N[C@@H](Cc1c[nH]cn1)C(=O)O] |

The second component is a biological material present in the human body and thus is very safe. Synthetic amine compounds exhibiting bad odors or toxicity are preferably excluded.

In the present invention, the first and second components contained as active ingredients in the deodorizing composition may chemically react with each other.

The first and second components may chemically react with each other in the deodorizing composition and then generate aminosulfites in the form of R₁—NHSO₂H.

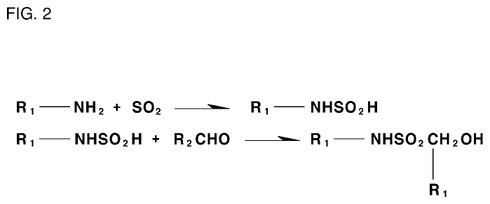

As shown in FIG. 2, the deodorizing mechanism is presumably the chemical reaction between the sulfurous acid generated in the first component, the amine group of the second component, and the aldehyde compound, as causes of smells.

In FIG. 2, R₁ in the second component is the chemical structure of a part excluding amino groups from an organic compound having an amino group. Specifically, the second component is one of compounds having at least one amino group, selected from the group consisting of taurine, adenosine, cytosine, thymine, adenine, guanine, lysine, arginine and histidine, but not limited thereto.

In addition, R₂ in FIG. 2 is an alkyl group in aldehyde compounds causing an odor, and the aldehyde compounds are specifically a chemical structure of one or more compounds selected from the group consisting of butanal, pentanal, nonenal, hexanal, heptanal, octanal, decanal and nonanal, which are substances causing hydrophobic aldehyde-based odors, but are not limited thereto.

As shown in FIG. 2, in the deodorizing composition of the present invention, the first component, sulfite, reacts with a compound having an amine group as the second component to produce amino sulfinic acid, which may react with an aldehyde-based compound. Since the product obtained through the reaction is highly hydrophilic and is easily soluble in water, it can diffuse into the aqueous layer during the extraction process and can be easily washed with a detergent.

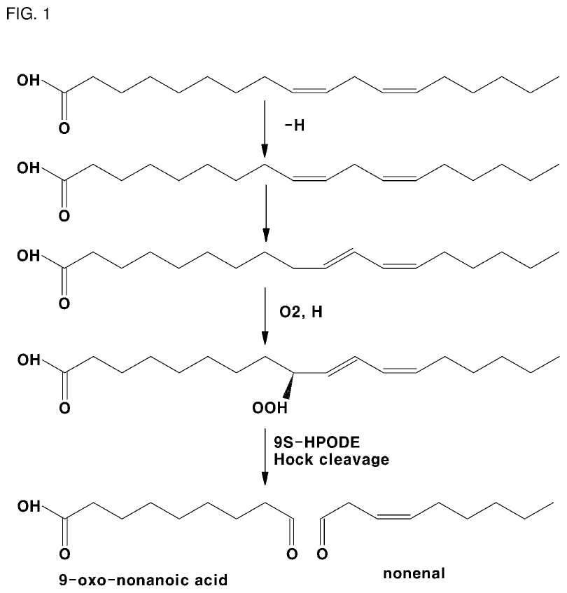

Thus, the deodorizing composition of the present invention, which includes the first and second components as active ingredients, can effectively remove aldehyde-based odors through the chemical reaction mechanism shown in FIG. 1.

The first and second components react with each other at an equivalent ratio, that is, a molar ratio. Accordingly, for example, when the amount of the first component is determined, the amount of the second component mixed therewith can be determined depending on the equivalent of the first component.

The second component may be mixed in a molar ratio of 1:1 to 1.5, 1:1 to 1.4, 1:1 to 1.3, or 1:1.2 with respect to the first component, but is not limited thereto.

In addition, the second component is mixed for reaction in an amount of 10 to 90% by weight, 8 to 80% by weight, 7 to 70% by weight, 6 to 60% by weight, or 5 to 50% by weight, based on the total weight of the first component and the second component, but is not limited thereto.

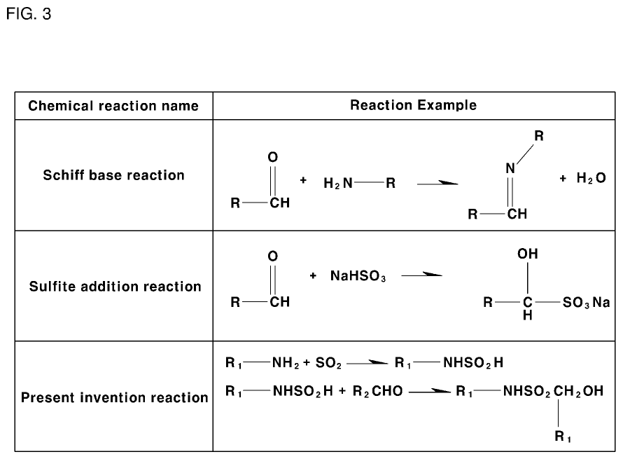

The mechanism of the chemical reaction between the aldehyde-based compounds and each of the first and second components and the mechanism of the chemical reaction between the aldehyde-based compound and the deodorizing composition containing both the first component and the second component as active ingredients according to the present invention are shown in FIG. 3.

The first component including one or more sulfites is capable of removing an aldehyde-based odor through a chemical addition reaction with an aldehyde-based compound, and the second component including a compound having at least one amine group can remove an aldehyde-based odor by generating an imine or an oxime compound by a Schiff base reaction between an amino acid having an amino group (NH₂) and an aldehyde-based compound.

In one embodiment of the present invention, the chemical reactions for the removal of the aldehyde-based compound by each of the first component and the second component through sulfite addition or Schiff base reactions (Comparative Examples 1 to 20) were found to be not more reactive than the chemical reactions for the removal of the aldehyde-based compound (Examples 1 to 30) by the deodorizing compositions containing both the first and second components as active ingredients (see Tables 5 and 6).

In the chemical reaction between the aldehyde-based compound and the deodorizing composition containing both the first and second components as active ingredients according to the present invention, the sulfurous acid produced in the first component simultaneously reacts with the amino group of the second component with a mechanism that is different from the Schiff base reaction derived from the second component and the sulfite addition reaction derived from the first component.

In one aspect, the aldehyde-based odor may be a hydrophobic aldehyde-based odor generated by evaporated chemicals with hydrophobic and aldehyde groups.

The substance causing the hydrophobic aldehyde-based odor includes, but is not limited to, at least one selected from the group consisting of butanal, pentanal, nonenal, hexanal, heptanal, octanal, decanal and nonanal.

In one embodiment of the present invention, when using a combination of the chemically reacted first and second components as active ingredients of the deodorizing composition for removing aldehyde-based odors, the deodorizing action is notably better than when using either the first or second components, which are capable of removing aldehyde-based odors, through addition or Schiff base reactions, respectively.

Specifically, compared to when nonenal is removed through the sulfite addition or Schiff-base reactions using the first or second components alone (Comparative Examples 1 to 19), better nonenal removal ability, that is, a notably excellent deodorizing effect, can be obtained when sulfite compounds as the first component are used in combination with biological compounds having primary or secondary amines as the second component (Experimental Example 1, Experimental Example 2, Table 5, and Table 6).

In an embodiment, the deodorizing composition containing the first and second components as active ingredients may have the ability to remove reactive oxygen species.

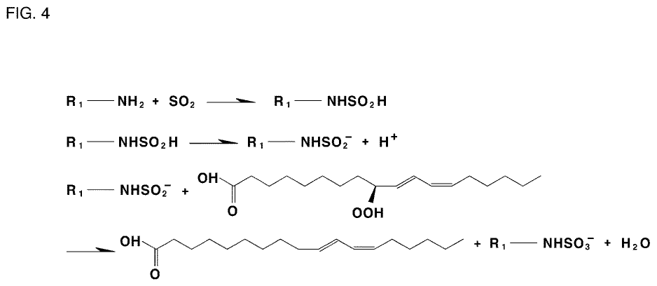

As shown in FIG. 4, the ability to remove reactive oxygen species can be obtained when peroxides, that is, reactive oxygen species, produced from unsaturated fatty acids through oxidation, react with amino sulfinic acid produced by the deodorizing composition and are thus removed and reduced.

In FIG. 4, R₁ is a chemical structure of a part excluding an amino group from an organic compound having the amino group. Specifically, R₁ may be a chemical structure of a part excluding an amino group from an organic compound having at least one amino group, and wherein the organic compound is selected from the group consisting of taurine, adenosine, cytosine, thymine, adenine, guanine, lysine, arginine and histidine as the second component, but is not limited thereto.

In an embodiment of the present invention, in order to investigate the ability to remove reactive oxygen species, of the deodorizing composition prepared by mixing the first component with the second component, the deodorizing composition prepared as shown in the Preparation Example 1 was vigorously mixed with omega-3 oil, which generates peroxides through oxidation, and was then removed from the omega-3 oil. The peroxide value of the omega-3 oil treated with the odorizing composition was measured and compared with that of the omega-3 oil before the treatment, as a control. The result showed that the peroxide value decreases; that is, the ability to remove reactive oxygen species was excellent (Experimental Example 3 and Table 7).

As described above, the deodorizing composition of the present invention having excellent deodorizing performance, which is prepared by mixing a sulfite compound as the first component in combination with a biological compound having a primary or secondary amine as the second component, can be used as a final product such as a deodorizer, fragrance or deodorant.

When producing the final product, the deodorizing composition of the present invention shows excellent deodorization capability in the state of an aqueous solution, but may be applied in a dry state or in the state of being mixed with other organic solvents depending on the situation. Accordingly, the concentration is not limited to a certain level, and can be arbitrarily adjusted depending on the application.

In addition, the deodorizing composition of the present invention may be added to final products such as household products. Examples of the household products include deodorant sprays, detergents for clothes, bleaches for clothes, softeners for clothes, detergents for dishes, cleaners for bathrooms, cleaners for toilets, and the like.

The content of the deodorizing composition of the present invention in the household products is not strictly limited, and depends on the odor intensity, and may be 0.001 to 10% by weight or 0.001 to 5% by weight based on the total weight of the household products.

The deodorizing composition of the present invention may be used to remove odors from food waste, clothing, refrigerators, closets/wardrobes/lockers, indoor areas, vehicles, toilets, bathrooms, pet supplies, factories, industrial wastewater, air cleaners, air conditioners, deodorizers, filters for blowers and ventilators, sewage treatment plants, livestock, and garbage treatment plants.

In another aspect, the present invention provides food additives containing the deodorizing composition to remove bad smells.

When the deodorizing composition of the present invention is used as food additives, the deodorizing composition may be added alone or in combination with other food ingredients, and may be suitably used in accordance with conventional methods. The amounts of the deodorizing composition mixed in foods can be appropriately determined according to the application thereof. Generally, the deodorizing composition of the present invention is added in an amount of 10 parts by weight or less, preferably 5 parts by weight or less, based on the weight of the raw materials. However, for long-term intake, the amount may be below the range defined above. The deodorizing composition may not be used in an amount lower than the range defined above.

There are no particular restrictions on application to foods. Examples of foods that can be treated with the deodorizing composition of the present invention to remove bad smells include unsaturated fatty acids, including omega-3, squalene, fish oil, flaxseed oil, hempseed oil, evening primrose oil, linoleic acid, olive oil, safflower seed oil, corn oil, soybean oil, and fish oil. The treated oils may be applied to meats, sausages, breads, chocolates, candies, snacks, confectioneries, pizzas, ramen, other kinds of noodles, gums, and dairy products such as ice creams, various soups, beverages, teas, drinks, alcoholic beverages and vitamin complexes, and foods in the conventional sense.

In addition, the deodorizing composition of the present invention can be applied as an aqueous solution, spray, dispensed solution, or aerosol, but is not limited thereto.

For example, the deodorizing composition can be used by washing or spraying to remove fishy smells of aldehyde compounds generated by the oxidation of oils in fishes. The deodorizing composition of the present invention may be used in mixtures with unsaturated oils such as omega-3, squalene, fish oil, flaxseed oil, hempseed oil, linoleic acid, olive oil, evening primrose oil and safflower seed oil to remove or reduce odors emitted from unsaturated oils.

Meanwhile, the deodorizing composition of the present invention is capable of removing oxidative rancidity as well as reactive oxygen species generated by the oxidation of unsaturated oils.

In another aspect, the present invention provides cosmetic additives containing the deodorizing composition to remove or reduce bad smells from the human body.

When the deodorizing composition of the present invention is used as a cosmetic additive, it may be used alone or in combination with cosmetically acceptable media or bases. The deodorizing composition may be provided as any formulation suitable for topical application, for example, solutions, gels, solids, anhydrous paste products, emulsions obtained by dispersing an oily phase in an aqueous phase, emulsions obtained by dispersing an aqueous phase in an oily phase, multi-emulsions, suspensions, micro-emulsions, micro-capsules, microgranules or ionic (liposomal) and nonionic vesicle dispersants, creams, skins, lotions, powders, ointments, sprays, cleansers and concealing sticks. In addition, the deodorizing composition according to the present invention may be used in the form of a foam, an aerosol composition containing a compressed propellant, or a patch. These compositions can be prepared according to conventional methods in the art.

There are no particular restrictions on the type of cosmetics. Examples of cosmetics to which an additive containing the deodorizing composition as an active ingredient can be applied include lotions, deodorants, solid soaps, liquid soaps, sprays, powdery perfumes, softening toner, nutrition toner, essences, nutrition creams, massage creams, packs, gels, makeup bases, foundations, powders, lipsticks, patches, sprays, eye creams, eye essences, cleansing creams, cleansing foams, cleansing water, cleansers, body lotions, body creams, body oils, body essences, shampoos, rinses, conditioners, treatments, hair packs, bleaching agents, permanent agents, hair dyes, and the like, and include all cosmetic products in the conventional sense for the purpose of removing odorous substances and reactive oxygen species from the skin, that is, for anti-aging.

In another aspect, the present invention provides a method for removing an aldehyde-based odor or reactive oxygen species using the deodorizing composition.

The deodorizing composition of the present invention, prepared by mixing a sulfite compound as the first component with an amine compound having amino group as the second component, is capable of simultaneously removing aldehydes and reactive oxygen species generated in foods, animals, the human body or the like. By spraying the composition to foods, animals, the human body and the like, bad smells can be removed.

In another aspect, the present invention provides a method for removing aldehyde-based odors including applying the deodorizing composition to or around a subject in need of odor removal.

The deodorizing composition of the present invention, prepared by mixing a sulfite compound as the first component with an amine compound having amino group as the second component, has excellent deodorizing performance on aldehyde-based odors, particularly hydrophobic aldehyde-based odors. An odorous substance such as an aldehyde can be effectively removed by directly applying the composition to foods, animals, human bodies, or the like containing an aldehyde-based odor component, or by applying the composition near a subject.

Hereinafter, the present invention will be described in more detail with reference to Examples, Experimental Examples and Preparation Examples. However, the following Examples, Experimental Examples and Preparation Examples are provided only for illustration of the present invention, and should not be construed as limiting the scope of the present invention. It will be obvious to those skilled in the art that a variety of modifications and alterations are possible without departing from the ideas and scope of the present invention.

### EXAMPLE

### Examples 1 to 20

In order to develop a bio-friendly deodorant capable of removing hydrophobic aldehyde compounds and reactive oxygen species, a deodorizing composition was prepared using only a substance that is permitted to be used as a cosmetic or food ingredient for application to fields involving close contact with humans, such as cosmetics and food.

Specifically, as shown in Table 1 below, the first components used herein were sulfite compounds, specifically sodium bisulfite, potassium bisulfite, lithium bisulfite, sodium metabisulfite, potassium metabisulfite, sodium metabisulfite, lithium metabisulfite, sodium hydrosulfite, potassium hydrosulfite, and lithium hydrosulfite, and the second components used herein were biological materials having primary or secondary amine compounds, such as taurine, adenosine, cytosine, thymine, adenine, guanine, lysine, arginine and histidine. The first and second components were mixed such that the second components were contained at a higher equivalent ratio than the first components; specifically, the first and second components were mixed at the equivalent ratio of 5:6, and the resulting mixtures were dissolved in purified water to prepare deodorizing solutions.

TABLE 1
Deodorizing compositions according to Examples 1 to 20.

| | | | Second | | Distilled |
|---|---|---|---|---|---|
| | First component | | component | | water |
| Example | Component | mmol | Component | mmol | (ml) |
| Example 1 | Sodium bisulfite | 5 | Taurine | 6 | 100 |
| Example 2 | Sodium bisulfite | 5 | Adenosine | 6 | 100 |
| Example 3 | Potassium | 5 | Cytosine | 6 | 100 |
| | bisulfite | | | | |
| Example 4 | Lithium bisulfite | 5 | Thymine | 6 | 100 |
| Example 5 | Sodium | 5 | Adenine | 6 | 100 |
| | metabisulfite | | | | |
| Example 6 | Potassium | 5 | Guanine | 6 | 100 |
| | metabisulfite | | (guanine) | | |
| Example 7 | Sodium | 5 | Lysine | 6 | 100 |
| | metabisulfite | | | | |
| Example 8 | Lithium | 5 | Arginine | 6 | 100 |
| | metabisulfite | | | | |
| Example 9 | Sodium | 5 | Histidine | 6 | 100 |
| | hydrosulfite | | | | |
| Example 10 | Potassium | 5 | Lysine | 6 | 100 |
| | hydrosulfite | | | | |
| Example 11 | Lithium | 5 | Lysine | 6 | 100 |
| | hydrosulfite | | | | |
| Example 12 | Sodium bisulfite | 5 | Histidine | 6 | 100 |
| Example 13 | Potassium | 5 | Arginine | 6 | 100 |
| | bisulfite | | | | |
| Example 14 | Lithium bisulfite | 5 | Lysine | 6 | 100 |
| Example 15 | Sodium | 5 | Guanine | 6 | 100 |
| | metabisulfite | | | | |
| Example 16 | Potassium | 5 | Adenine | 6 | 100 |
| | metabisulfite | | | | |
| Example 17 | Sodium | 5 | Thymine | 6 | 100 |
| | metabisulfite | | | | |
| Example 18 | Lithium | 5 | Cytosine | 6 | 100 |
| | metabisulfite | | | | |
| Example 19 | Sodium | 5 | Taurine | 6 | 100 |
| | hydrosulfite | | | | |
| Example 20 | Potassium | 5 | Histidine | 6 | 100 |
| | hydrosulfite | | | | |

### Examples 21 to 25

In Examples 1 to 20, the deodorizing composition was prepared by mixing a single compound as the first component with a single compound as the second component. In addition, in Examples 21 to 25, deodorizing compositions were prepared using the same equivalent ratio as in Examples 1 to 20 (first component:second component=5:6) and dissolved in the same amount of solvent (purified water 100 ml), wherein the first and second components were mixed as shown in Table 2. The effect of the mixtures of the first and second components on the deodorization performance was analyzed as follows.

Specifically, based on the results of anisidine values of test materials treated with deodorizing compositions of Examples 1 to 20 and Comparative Examples 1 to 20, anisidine values of test materials treated with Examples 1 to 20 were lower than those of Comparative Examples 1 to 20 (see Table 5), and because the anisidine value expresses the amount of aldehyde compounds in a test material, a lower anisidine value means a lower amount of aldehyde compounds in the oil. Therefore, the deodorizing compositions according to Examples 1 to 20, which have lower anisidine values, were more effective than those of Comparative Examples 1 to 20. At this time, the molar ratio of the first and second components was 5:6 (mmol/mmol) and the compounds in each component were used in identical molar amounts. In addition, the amount of purified water used as a solvent in each example was 100 ml.

TABLE 2
Deodorizing compositions according to Examples 21 to 25

| | First component | | Second component | |
|---|---|---|---|---|
| Example | Component | Component | Component | Component |
| Example 21 | Sodium | Potassium | Taurine | Adenine |
| | bisulfite | bisulfite | | |
| Example 22 | Sodium | Lithium | Adenine | Arginine |
| | metabisulfite | hydrosulfite | | |
| Example 23 | Lithium | lithium | Arginine | Lysine |
| | metabisulfite | bisulfite | | |
| Example 24 | Lithium | Sodium | Lysine | Histidine |
| | hydrosulfite | bisulfite | | |
| Example 25 | Potassium | Sodium | Arginine | Lysine |
| | bisulfite | hydrosulfite | | |

### Examples 26 to 30

In Examples 1 to 25, the first and second components were mixed at concentrations of 5 and 6 mmol, respectively. Meanwhile, in Examples 26 to 30, the first and second components were mixed at concentrations of 10 and 12 mmol. The concentrations of first and second components in Examples 26 to 30 increased 2 times than those in Examples 1 to 25 to investigate the deodorizing effect.

Specifically, as shown in Table 3, the first and second components were dissolved in 100 ml of purified water at concentrations of 10 and 12 mmol (molar ratio of 10:12), respectively.

TABLE 3
Deodorizing compositions according to Examples 26 to 30.

| | | | | | Distilled |
|---|---|---|---|---|---|
| | First component | | Second component | | water |
| Example | Component | mmol | Component | mmol | (ml) |
| Example 26 | Sodium bisulfite | 10 | Taurine | 12 | 100 |
| Example 27 | Sodium metabisulfite | 10 | Adenine | 12 | 100 |
| Example 28 | Lithium metabisulfite | 10 | Arginine | 12 | 100 |
| Example 29 | Lithium hydrosulfite | 10 | Lysine | 12 | 100 |
| Example 30 | potassium bisulfite | 10 | Arginine | 12 | 100 |

### Comparative Examples 1 to 20

Aqueous solutions containing only the first component or the second component were prepared as control groups for comparison with the deodorizing performance of Examples 1 to 20, and the compositions thereof are shown in Table 4.

TABLE 4
Deodorizing compositions
according to Comparative Examples 1 to 20

| | | | | | Distilled |
|---|---|---|---|---|---|
| Comparative | First component | | Second component | | water |
| Example | Component | mmol | Component | mmol | (ml) |
| Comparative | Sodium | 5 | — | — | 100 |
| Example 1 | bisulfite | | | | |
| Comparative | Potassium | 5 | — | — | 100 |
| Example 2 | bisulfite | | | | |
| Comparative | Lithium | 5 | — | — | 100 |
| Example 3 | bisulfite | | | | |
| Comparative | Sodium | 5 | — | — | 100 |
| Example 4 | metabisulfite | | | | |
| Comparative | Potassium | 5 | — | — | 100 |
| Example 5 | metabisulfite | | | | |
| Comparative | Sodium | 5 | — | — | 100 |
| Example 6 | metabisulfite | | | | |
| Comparative | Lithium | 5 | — | — | 100 |
| Example 7 | metabisulfite | | | | |
| Comparative | Sodium | 5 | — | — | 100 |
| Example 8 | hydrosulfite | | | | |
| Comparative | Potassium | 5 | — | — | 100 |
| Example 9 | hydrosulfite | | | | |
| Comparative | Lithium | 5 | — | — | 100 |
| Example 10 | hydrosulfite | | | | |
| Comparative | — | — | Taurine | 6 | 100 |
| Example 11 | | | | | |
| Comparative | — | — | Adenosine | 6 | 100 |
| Example 12 | | | | | |
| Comparative | — | — | Cytosine | 6 | 100 |
| Example 13 | | | | | |
| Comparative | | | Thymine | 6 | 100 |
| Example 14 | — | — | | | |
| Comparative | | | Adenine | 6 | 100 |
| Example 15 | — | — | | | |
| Comparative | | | Guanine | 6 | 100 |
| Example 16 | — | — | | | |
| Comparative | | | Lysine | 6 | 100 |
| Example 17 | | | | | |
| Comparative | — | — | Arginine | 6 | 100 |
| Example 18 | | | | | |
| Comparative | — | — | Histidine | 6 | 100 |
| Example 19 | | | | | |
| Comparative | — | — | — | — | 100 |
| Example 20 | | | | | |

### Experimental Example 1: Analysis of Deodorization Performance of Compositions According to Examples 1 to 20

In order to determine the deodorizing performance of the compositions according to Examples 1 to 20 and Comparative Examples 1 to 20, the anisidine value (p-AV), used to measure the content of aldehyde compounds as bad odor sources, which are generated by the decomposition of peroxides, was analyzed using ultraviolet spectrometry. At this time, 0.2% 2-nonenal isooctane solution (v/v) was used as a standard solution to determine the deodorizing performance. 2-Nonenal is a typical aldehyde compound generated by the oxidation of unsaturated fatty acids in human skin. 20 ml of the standard solution was placed in a container containing 20 ml of the composition (that is, a deodorant substance) of each of Examples 1 to 20 and Comparative Examples 1 to 20, and then vigorously stirred for 30 minutes to allow nonenal in isooctane to react with the deodorant substance in water and thereby remove nonenal. The deodorizing performance of the deodorizing substances was determined by measuring the anisidine values (p-AV).

As shown in Table 5, the deodorizing composition of Comparative Example 20, in which the component was water (purified water), had a p-AV almost identical to that of a 0.2% nonenal standard solution, that is, had no deodorizing effect. This suggests that the deodorizing effect is not exhibited since nonenal is hydrophobic and thus is insoluble in water. In addition, the deodorizing compositions of Comparative Examples 1 to 19, using a single substance, were also shown to have high p-AV similar to that of water and thus were thought to have little deodorizing performance.

Meanwhile, when using the deodorizing compositions of Examples 1 to 20, prepared by mixing sulfite compounds as the first component and biological compounds having primary or secondary amines as the second component, p-AV was relatively low compared to the deodorizing compositions of Comparative Examples 1 to 20. Therefore, this demonstrates that, when using the combination of sulfite and amine compounds, better nonenal removal ability, that is, a remarkably excellent deodorizing effect, can be obtained compared to sulfite addition and Schiff base reactions using either the first or second components (Comparative Examples 1 to 19).

In particular, the deodorizing compositions containing lithium bisulfite and lysine (Example 14), potassium metabisulfite and guanine (Example 6), sodium metabisulfite and adenine (Example 5), potassium bisulfite and arginine (Example 13), and lithium hydrosulfite and lysine (Example 11) had remarkably excellent deodorizing effects.

TABLE 5
Anisidine values after reactions of deodorizing compositions according to
Examples 1 to 20 and Comparative Examples 1 to 20

| | Anisidine | | Anisidine |
|---|---|---|---|
| Example | value (p-AV) | Comparative Example | value (p-AV) |
| Example 1 | 22.12 | Comparative Example 1 | 33.28 |
| Example 2 | 25.23 | Comparative Example 2 | 33.23 |
| Example 3 | 24.65 | Comparative Example 3 | 34.65 |
| Example 4 | 23.48 | Comparative Example 4 | 34.48 |
| Example 5 | 15.78 | Comparative Example 5 | 33.10 |
| Example 6 | 14.58 | Comparative Example 6 | 35.36 |
| Example 7 | 23.59 | Comparative Example 7 | 34.27 |
| Example 8 | 22.35 | Comparative Example 8 | 34.39 |
| Example 9 | 23.71 | Comparative Example 9 | 34.10 |
| Example 10 | 24.11 | Comparative Example 10 | 33.98 |
| Example 11 | 16.59 | Comparative Example 11 | 35.98 |
| Example 12 | 26.57 | Comparative Example 12 | 36.72 |
| Example 13 | 15.88 | Comparative Example 13 | 37.21 |
| Example 14 | 13.37 | Comparative Example 14 | 36.54 |
| Example 15 | 25.61 | Comparative Example 15 | 36.23 |
| Example 16 | 26.28 | Comparative Example 16 | 38.45 |
| Example 17 | 25.71 | Comparative Example 17 | 34.56 |
| Example 18 | 17.22 | Comparative Example 18 | 38.41 |
| Example 19 | 18.32 | Comparative Example 19 | 38.09 |
| Example 20 | 18.45 | Comparative Example 20 | 37.73 |
| | | 0.2% nonenal standard solution | 36.48 |

### Experimental Example 2: Analysis of Deodorization Performance of Deodorizing Compositions According to Examples 21 to 25 and Examples 26 to 30

In order to determine the deodorizing performance of the deodorizing compositions according to Examples 21 to 25 and Examples 26 to 30, the anisidine values were analyzed in the same manner as in Experimental Example 1.

As shown in Table 6, the deodorizing performance of the deodorizing compositions according to Examples 21 to 25, prepared by mixing a combination of two compounds as the first component with a combination of two compounds as the second component, was similar to the deodorizing performance of the deodorizing compositions according to Examples 1 to 20, prepared by mixing a single compound as the first component with a single compound as the second component. Thus, it was found that, although the deodorizing composition was prepared by mixing the first and second components to achieve a combined form, the anisidine values were not significantly affected.

Meanwhile, the deodorizing performance of the compositions according to Examples 26 to 30 increased the concentration of each first and second component by 2 times, compared to Examples 1 to 20, which is a remarkable improvement.

TABLE 6
Anisidine values after reactions of deodorizing compositions
according to Examples 21 to 25 and Examples 26 to 30

| Example | Anisidine value (p-AV) |
|---|---|
| Example 21 | 22.37 |
| Example 22 | 17.56 |
| Example 23 | 16.50 |
| Example 24 | 18.32 |
| Example 25 | 17.11 |
| Example 26 | 5.72 |
| Example 27 | 6.54 |
| Example 28 | 7.42 |
| Example 29 | 7.89 |
| Example 30 | 9.21 |

### Experimental Example 3: Analysis of Ability of Deodorizing Composition to Remove Reactive Oxygen Species

In order to determine the ability to remove reactive oxygen species of the deodorizing compositions in Examples, the peroxide values of a starting material (omega-3 oil manufactured by AK & MN Biofarm) and omega-3 prepared according to Preparation Example 1 were measured using the method in the Korean Food Standards Codex. As shown in Table 7, the peroxide value was reduced, that is, the ability to remove reactive oxygen species was excellent.

TABLE 7
Peroxide value of omega-3 oil treated with Preparation
Example 1, compared to raw oil.

| | | | Peroxide |
|---|---|---|---|
| Sample name | Unit | Test method | value |
| Raw omega-3 | meq | Korean Food | 15.5 |
| | (milliequivalent)/kg | Standards Codex | |
| Preparation | meq/kg | Korean Food | 8.7 |
| Example 1 | | Standards Codex | |

The series of the results described above demonstrated that the deodorizing compositions of the present invention, prepared by mixing sulfite compounds as the first component and biological compounds having primary or secondary amines as the second component, exhibit excellent deodorizing performance as well as excellent ability to remove reactive oxygen species.

Accordingly, the deodorizing composition of the present invention can remove odorous substances such as aldehydes and reactive oxygen species generated from foods, animals, human bodies and the like, and thus can be useful as a bio-friendly additive to impart deodorizing and anti-aging functions to foods or cosmetics.

### Preparation Example 1: Omega-3

In order to prepare odorless omega-3 oil, a deodorizing aqueous solution was prepared using lithium bisulfite and lysine, shown in Table 5, having the best deodorizing effect among the Examples. Specifically, 0.10 mol of lithium bisulfite and 0.11 mol of lysine were added to 50 ml of distilled water to prepare a deodorizing aqueous solution, and then 50 ml of omega-3 oil was added to the aqueous solution and stirred vigorously in a reactor for 10 min to remove an aldehyde-based odorous substance contained in omega-3 oil. After water and oil layers separated, omega-3 oil was recovered, and then a small amount of anhydrous calcium sulfate was added thereto. The mixture was stirred slowly to remove residual water in the oil and then calcium sulfate was removed from the oil. Soft capsules were produced with odorless omega-3 oil from which odorous aldehyde compounds were removed in this Example.

### Preparation Example 2: Squalene

In order to prepare odorless squalene, 0.10 mol of potassium metabisulfite as the first component and 0.11 mol of histidine as the second component were added to 50 ml of distilled water to prepare a deodorizing aqueous solution. Then, 50 ml of squalene was added to the aqueous solution and stirred vigorously in a reactor for 10 min to remove aldehyde-based odorous substances contained in squalene. After water and oil layers separated, squalene was recovered, and a small amount of anhydrous calcium sulfate was added thereto. The mixture was stirred slowly to remove residual water in the oil, and then calcium sulfate was removed from the oil. Soft capsules were produced with odorless squalene from which odorous aldehyde compounds were removed through this Example.

### Preparation Example 3: Spray Formulation

In order to prepare a spray formulation that can be used to remove fishy smells from fishes or kitchen utensils and to remove odors from laundry. The deodorizing composition in Example 1 containing 5 mmol of sodium bisulfite and 6 mmol of taurine was diluted 2 times with ethanol to prepare an ethanol solution. The solution was placed in a spray container for use as a deodorant for removing bad odors from fishes, laundry, etc.

### Preparation Example 4: Deodorant

In order to prepare a deodorant product for preventing body odor in humans and animals, the deodorizing composition of Example 13, containing 5 mmol of potassium bisulfite and 6 mmol of arginine, was diluted 2 times to prepare 100 ml of the deodorant solution. Then, 20 ml of ethanol and 0.2 ml of a rosemary essence were added to prepare the spray formulation. The spray formulation is capable of being used as a deodorant to remove bad odors such as nonenal from the human body, clothes and bedding, and also to remove bad odors from animals such as pets.

### Preparation Example 5: Lotion

In order to prepare a lotion for removing body odors, 3 g of olive wax, 15 g of olive oil, 3 g of glycerin and 0.2 ml of rosemary extract were added to 80 ml of a deodorizing solution prepared by diluting 4 times the deodorizing composition of Example 6 containing 5 mmol of potassium metabisulfite and 6 mmol of guanine, followed by stirring for 20 to 30 minutes with a homomixer while heating (60-80° C.), to prepare a lotion as a skin care product.

### Preparation Example 6: Deodorizing Soap

The components shown in Table 8 were mixed at a temperature of 40° C. to prepare deodorizing soaps for removing nonenal, which is the source of bad smells in the elderly, from the skin.

TABLE 8
Preparation Examples of deodorizing soaps

| | Deodorizing | Deodorizing | Deodorizing |
|---|---|---|---|
| Component | soap 1 | soap 2 | soap 3 |
| Coconut oil | 25 g | 25 g | 25 g |
| Soybean oil | 55 g | 55 g | 55 g |
| Olive oil | 5 g | 5 g | 5 g |
| NaOH | 12 g | 12 g | 12 g |
| Glycerin | 3 g | 3 g | 3 g |
| Sulfites | Potassium | Lithium | Sodium |
| | metabisulfite | bisulfite | hydrosulfite |
| | 0.5 g | 1 g | 2 g |
| Amines | Lysine 1 g | Histidine 1.5 g | Guanine 3 g |
| Fragrances | Rosemary | Rosemary | Rosemary |
| | extract 0.5 g | extract 0.5 g | extract 0.5 g |
| Distilled water | 25 g | 25 g | 25 g |
| (Water) | | | |

### Preparation Example 7: Deodorizing Liquid Soap

The components shown in Table 9 were mixed at a temperature of 40° C. to prepare deodorizing liquid soaps for removing nonenal from the skin.

TABLE 9
Preparation Examples of deodorizing liquid soaps

| | Deodorizing | Deodorizing | Deodorizing |
|---|---|---|---|
| | liquid | liquid | liquid |
| Component | soap 1 | soap 2 | soap 3 |
| Sodium lauryl ether | 12.0 g | 12.0 g | 12.0 g |
| sulfate (2 moles EO | | | |
| (ethylene oxide)) | | | |
| Sodium lauryl sulfate | 3.3 g | 3.3 g | 3.3 g |
| Cocamide MEA | 5.5 g | 5.5 g | 5.5 g |
| Guar hydroxypropyl- | 0.15 g | 0.15 g | 0.15 g |
| trimonium chloride | | | |
| Tetrasodium ethylene- | 0.08 g | 0.08 g | 0.08 g |
| diaminetetraacetate | | | |
| Glycol stearate | 0.75 g | 0.75 g | 0.75 g |
| Stearamide MEA | 2.0 g | 2.0 g | 2.0 g |
| PEG 150 distearate | 0.25 g | 0.25 g | 0.25 g |
| Castor oil | 0.5 g | 0.5 g | 0.5 g |
| Stearamidopropyl | 0.2 g | 0.2 g | 0.2 g |
| pg-dimonium | | | |
| chloride phosphate | | | |
| Cocamidopropyl | 1.9 g | 1.9 g | 1.9 g |
| hydroxysultaine | | | |
| Sodium chloride | 0.18 g | 0.18 g | 0.18 g |
| Sulfites | Sodium | Potassium | Lithium |
| | hydrosulfite | hydrosulfite | hydrosulfite |
| | 2 g | 2 g | 2 g |
| Amines | Adenine | Thymine | Guanine |
| | 3 g | 3 g | 3 g |
| Rosemary extract | 0.3 g | 0.3 g | 0.3 g |
| Hydroxypropyl | 0.1 g | 0.1 g | 0.1 g |
| methylcellulose (HPMC) | | | |
| Distilled water (Water) | 100 ml | 100 ml | 100 ml |

### Preparation Example 8: Soybean Oil

In order to prepare odorless soybean oil, 0.10 mol of potassium metabisulfite and 0.11 mol of histidine were added to 50 ml of distilled water to prepare a deodorizing aqueous solution. Then, 50 ml of raw soybean oil was added to the aqueous solution and stirred vigorously in a reactor for 10 min to remove an aldehyde-based odorous substance contained in the soybean oil. After stopping the reactor and separating water and oil layers, soybean oil was recovered, and then a small amount of anhydrous calcium sulfate was added thereto, and the mixture was stirred slowly to remove residual water to prepare purified soybean oil.

### Preparation Example 9: Grapeseed Oil

In order to prepare odorless grapeseed oil, 0.10 mol of potassium metabisulfite and 0.20 mol of lysine were added to 50 ml of distilled water to prepare the deodorizing aqueous solution. Then, 50 ml of raw grapeseed oil was added to the aqueous solution and stirred vigorously in a reactor for 10 min to remove the aldehyde-based odorous substances contained in the grapeseed oil. After stopping the reactor and separating water and oil layers, grapeseed oil was recovered, and then a small amount of anhydrous calcium sulfate was added thereto, and the mixture was stirred slowly to remove residual water to prepare the purified grapeseed oil. The deodorizing composition according to an aspect of the present invention has an effect of removing reactive oxygen species as well as an effect of removing substances causing aldehyde-based odors, particularly hydrophobic aldehyde-based odors.

Thus, by applying food additives containing the deodorizing compositions according to the aspect of the present invention, an effect of removing odors from food can be obtained.

In addition, by applying cosmetic additives containing the deodorizing compositions according to the aspect of the present invention, effects of reducing bad body odors from humans and animals and removing reactive oxygen species, thereby providing the anti-aging function can be obtained.

The effects of the present invention are not limited to those described above. It should be understood that the effects of the present invention include all effects that can be inferred from the description above.

The present invention has been described in detail with reference to preferred embodiments thereof. However, it will be appreciated by those skilled in the art that changes may be made in these embodiments without departing from the principles and spirit of the invention, the scope of which is defined in the appended claims and their equivalents.

What is claimed is:

1. A deodorizing composition comprising, as active ingredients, a first component comprising at least one sulfite compound and a second component comprising at least one amine compound having one or more amino groups.

2. The deodorizing composition according to claim 1, wherein the first component comprises at least one selected from the group consisting of sodium bisulfite, potassium bisulfite, lithium bisulfite, sodium metabisulfite, potassium metabisulfite, sodium metabisulfite, lithium metabisulfite, sodium hydrosulfite, potassium hydrosulfite, and lithium hydrosulfite.

3. The deodorizing composition according to claim 1, wherein the second component comprises at least one selected from the group consisting of taurine, adenosine, cytosine, thymine, adenine, guanine, lysine, arginine, and histidine.

4. The deodorizing composition according to claim 1, wherein the first component and the second component react with each other in the composition, and thus are present as R₁—NHSO₂H, wherein R₁ is a part excluding an amino group from a compound having an amino group.

5. The deodorizing composition according to claim 1, wherein the first component and the second component are mixed at a molar ratio of 1:1 to 1.5.

6. The deodorizing composition according to claim 1, wherein the deodorizing is removal of an aldehyde-based odor.

7. The deodorizing composition according to claim 6, wherein the aldehyde-based odor is a hydrophobic aldehyde-based odor.

8. The deodorizing composition according to claim 7, wherein a substance causing the hydrophobic aldehyde-based odor comprises at least one selected from the group consisting of butanal, pentanal, nonenal, hexanal, heptanal, octanal, decanal and nonanal.

9. The deodorizing composition according to claim 1, wherein the deodorizing composition has the ability to remove reactive oxygen species.

10. A food additive comprising the deodorizing composition according to claim 1 as an active ingredient.

11. A cosmetic additive comprising the deodorizing composition according to claim 1 as an active ingredient.

12. A method for removing aldehyde-based odors or reactive oxygen species using the deodorizing composition according to claim 1.

13. A method for removing aldehyde-based odors comprising applying the deodorizing composition according to claim 1 to or around a subject in need of odor removal.
